(19) United States
(12) Patent Application Publication
(10) Pub. No.: US 2026/0263642 A1
(43) Pub. Date: Sep. 10, 2026

(54) N-OXIDE BASED ZWITTERIONIC NEAR INFRARED FLUOROPHORE IMAGING AGENTS AND METHODS OF USE THEREFOR

(71) Applicant: Curadel Surgical Innovations, Inc., Bonita Springs, FL (US)

(72) Inventors: John V. Frangioni, Bonita Springs, FL (US); Hak Soo Choi, Needham, MA (US)

(73) Assignee: Curadel Surgical Innovations, Inc., Bonita Springs, FL (US)

(21) Appl. No.: 19/564,861

(22) Filed: Mar. 12, 2026

Related U.S. Application Data
(63) Continuation-in-part of application No. PCT/US2024/046117, filed on Sep. 11, 2024.

(51) Int. Cl. A61K 49/00

(52) U.S. Cl. CPC A61K 49/0032; A61K 49/0052; A61K 49/0056; A61K 2121/00

(57) ABSTRACT

In accordance with at least one aspect of this disclosure, there is provided an imaging agent, a including dye compound conjugated to an antigen specific targeting vector which can be particularly advantageous because their behavior in vivo can contribute to superior optical imaging properties, for example, by significantly increasing the solubility of the dye and/or targeting vector, increasing the target-to-background ratio of imaged tissues, leading to higher resolution imaging, and ultimately providing for better recognition of malignant tissue for resection and margin assessment and improved visualization during minimal invasive laparoscopic surgery, for example. A method of imaging tumor cells in a subject, can include, administering an imaging effective amount of an imaging agent according at least one embodiment of the invention; irradiating a region in the subject in which tumor cells are expected to be found with at a wavelength absorbed by the imaging agent; and detecting a signal from the imaging agent.

### CROSS-REFERENCE TO RELATED APPLICATIONS

This application is a Continuation-in-Part of PCT International Patent Application Serial No. PCT/US2024/046117, filed Sep. 11, 2024. This application claims priority to U.S. Provisional Patent Application Ser. No. 63/648,302 filed May 16, 2024, and U.S. Provisional Patent Application Ser. No. 63/656,858 filed Jun. 6, 2024. The disclosures of each of these applications are incorporated herein by reference.

This application is also related to U.S. Provisional Patent Application Ser. No. 63/538,038 filed Sep. 12, 2023, U.S. Utility patent application Ser. No. 18/640,169 filed Apr. 19, 2024, U.S. Utility patent application Ser. No. 18/732,982 filed Jun. 4, 2024, U.S. Utility patent application Ser. No. 18/641,097 filed Apr. 19, 2024, U.S. Utility patent application Ser. No. 18/641,149 filed Apr. 19, 2024, and U.S. Utility patent application Ser. No. 18/641,620 filed Apr. 19, 2024, The disclosures of each of these applications are also incorporated herein by reference.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Mar. 12, 2026, is entitled “1515138_110US4_SL.xml”, and is 6,230 bytes in size.

### FIELD OF THE INVENTION

The present invention relates to methods of optically imaging tissues or cells using zwitterionic imaging agents having compact N-oxide-based zwitterions that result in improved stability and solubility as compared to other near-infrared (NIR) fluorescent contrast agents.

### BACKGROUND OF THE INVENTION

Near infrared (NIR) fluorescence has potential importance in the medical field, particularly in diagnostics and image-guided surgery. However, the availability of suitable fluorophores as imaging agents has been a primary hindrance. To be clinically viable, the ideal NIR fluorophore should have both good optical properties and superior in vivo properties with respect to solubility, metabolism, biodistribution, and clearance. Known fluorophores tend to clear partially through the liver, which results in undesirable fluorescence throughout the gastrointestinal tract. And in some cases, known fluorophores suffer from insufficient solubility, particularly in saline solutions, resulting in more difficult and costly modes of administration.

In recent years, more advanced fluorophores have been developed which result in improved target-to-background. See, for example, U.S. Pat. Nos. 11,077,210, 9,687,567, 10,493,169, 10,201,621, and 10,478,512. Although these advanced fluorophores lower non-specific uptake in non-target tissue, they are relatively labile in blood, becoming metabolized in minutes to hours. As such, there remains a need for new and improved NIR fluorescent imaging agents with tumor- or diseased-tissue targeting and/or increased stability that can equilibrate rapidly between the intravascular and extravascular spaces, have minimal non-specific and off-target uptake, and are cleared efficiently, including by renal filtration.

Further, although prior fluorophores have had some success in targeting specific antigens and other markers which are expressed by a variety of cancer cell types, there remains a need for increased targeting of specific antigens to increase the specific uptake of the fluorophores to increase signal strength. For example, targeting vectors directed to prostate-specific membrane antigen (PSMA), bombesin receptors, somatostatin receptors, fibroblast activation protein (FAP) would provide fluorophores with increased signal for more accurate imaging of various tumors and benign but diseased tissues, as well as tumor stroma, and other diseases characterized by tissue remodeling. The imaging agents of the invention are directed toward these and other needs.

Improved solubility, particularly in saline solution, as well as increased targeting potential provides for better recognition of malignant tissue for resection and margin assessment as well as improved visualization during minimally-invasive laparoscopic surgery. Accordingly, there remains a need in the art for further increasing solubility and target-to-background ratios to assist practitioners in performing complete and accurate resections of cancerous tissues during surgery.

### SUMMARY OF THE INVENTION

The invention is based, at least in part, on the discovery that the inclusion of N-oxide based zwitterionic components can increase the solubility of zwitterionic fluorophores without reducing the stability or clearance afforded by other elements of the fluorophore design.

The imaging agents of the disclosure are particularly advantageous because their behavior in vivo is believed to contribute to superior solubility, superior optical imaging properties and, in some cases, superior stability. More specifically, the charge-balancing is believed to impart good biodistribution and clearance properties to the agents and reduce undesirable non-specific binding while the inclusion of the targeting ligand increases the time of circulation and prevents additional degradation after binding to target cells. These in vivo properties help improve administration and the target-to-background ratio of imaged tissues, leading to higher sensitivity and higher resolution imaging.

In one aspect, the disclosure provides an imaging agent dye comprising imaging agent, optionally conjugated to a targeting vector, the imaging agent dye having one or more N-oxide based zwitterionic groups.

In one embodiment of the imaging agent dye according to the disclosure, the charge-balanced imaging agent is an agent of formula (I):

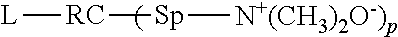

wherein for Formula (I):
L represents a linking group capable of conjugating to a targeting vector;
RC represents a resonant core;
Each Sp independently represents a spacer group; and
p represents an integer from 1-4.

In another embodiment of the imaging agent dye according to the disclosure, the imaging agent is an agent of formula (II):

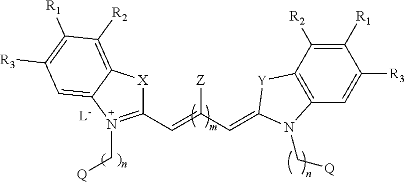

wherein for Formula (II):
each R₁ is independently —C₁-C₄ alkyl-N⁺(CH₃)₂O⁻;
each R₂ is independently H, —OR′, halogen, sulfonate, substituted or unsubstituted amino, C(O)NH—C₁-C₆ alkyl, C₁-C₆ alkyl, C₁-C₆ alkoxy or phenyl;
each R₃ is independently H, —OR′, halogen, sulfonate, substituted or unsubstituted amino, C(O)NH—C₁-C₆ alkyl, C₁-C₆ alkyl, C₁-C₆ alkoxy or phenyl;
   or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
   or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₃)₂O⁻;
X and Y are each independently O, S, Se, C(R″)₂, NR′″;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
each R′ is independently H, alkyl or aryl;
each R″ is independently H or alkyl;
each R′″ is independently H, akyl, akyl-SO₃H, or akyl-COOH;
m is an integer from 0-3; and
each n is independently an integer from 1-4; and
L is an anion;
or a salt, solvate, hydrate, polymorph, prodrug, or stereoisomer thereof,
and wherein the Z group is capable of conjugating a targeting vector.

In still another embodiment of the imaging agent dye according to the disclosure, the imaging agent is an agent of formula (III):

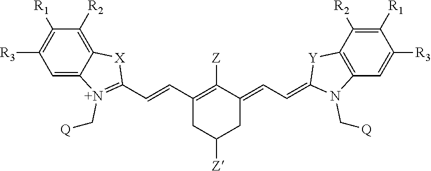

wherein for Formula (III):
each R₁ is independently —C₁-C₄ alkyl-N⁺(CH₃)₂O⁻;
each R₂ is independently H, —OR′, halogen, sulfonato, substituted or unsubstituted amino, C(O)NH—C₁-C₆ alkyl, C₁-C₆ alkyl, C₁-C₆ alkoxy or phenyl;
each R₃ is independently H, —OR′, halogen, sulfonato, substituted or unsubstituted amino, C(O)NH—C₁-C₆ alkyl, C₁-C₆ alkyl, C₁-C₆ alkoxy or phenyl;
   or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₂)₂O⁻ group, and, optionally, further substituted with halogen, alkyl, alkoxy, hydroxyl, —SO₂OH, or —CO₂H;
   or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₃)₂O⁻;
X and Y are each independently O, S, Se, C(R″)₂, NR;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
Z′ is H, or COOR;
R is independently H, —OR″″,
L is an anion;
each R′ is independently H, alkyl or aryl;
each R″ is independently H or alkyl;
each R′″ is independently H, akyl, akyl-SO₃H, or akyl-COOH;
each R″″ is independently H, akyl, aryl, NH₂, NHR, alkyl-NH₂, or alkyl-COOH;
each n is independently an integer from 1-4; and
L is an anion;
or a salt, solvate, hydrate, polymorph, prodrug, or stereoisomer thereof;
and wherein the Z group is capable of conjugating a targeting vector.

In yet another embodiment of the imaging agent dye according to the disclosure, the imaging agent is:

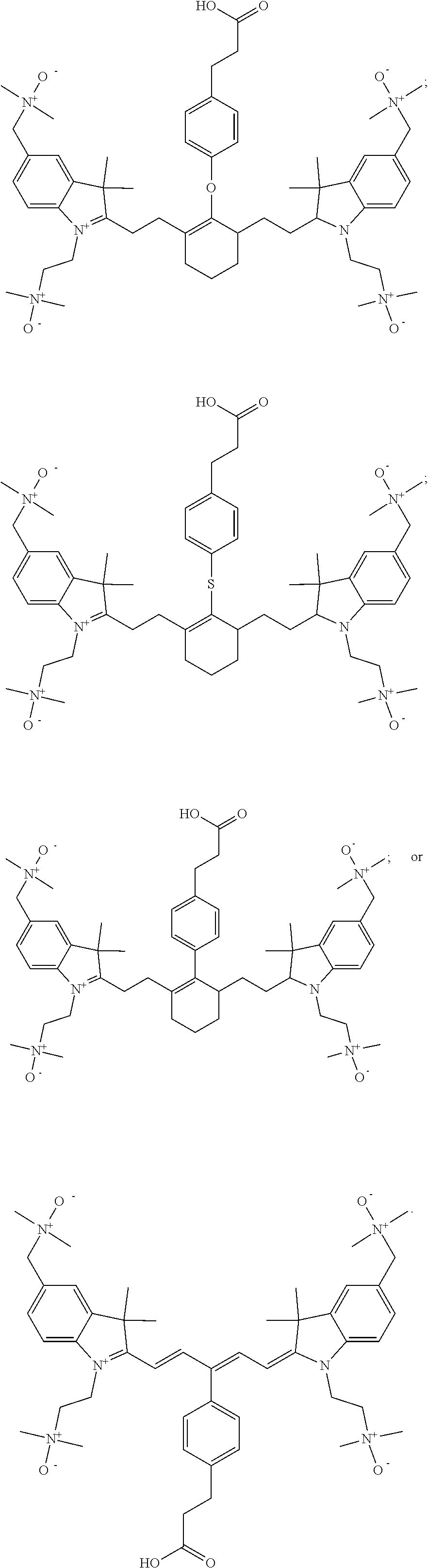

In yet another embodiment of the imaging agent dye according to the disclosure, the imaging agent is:

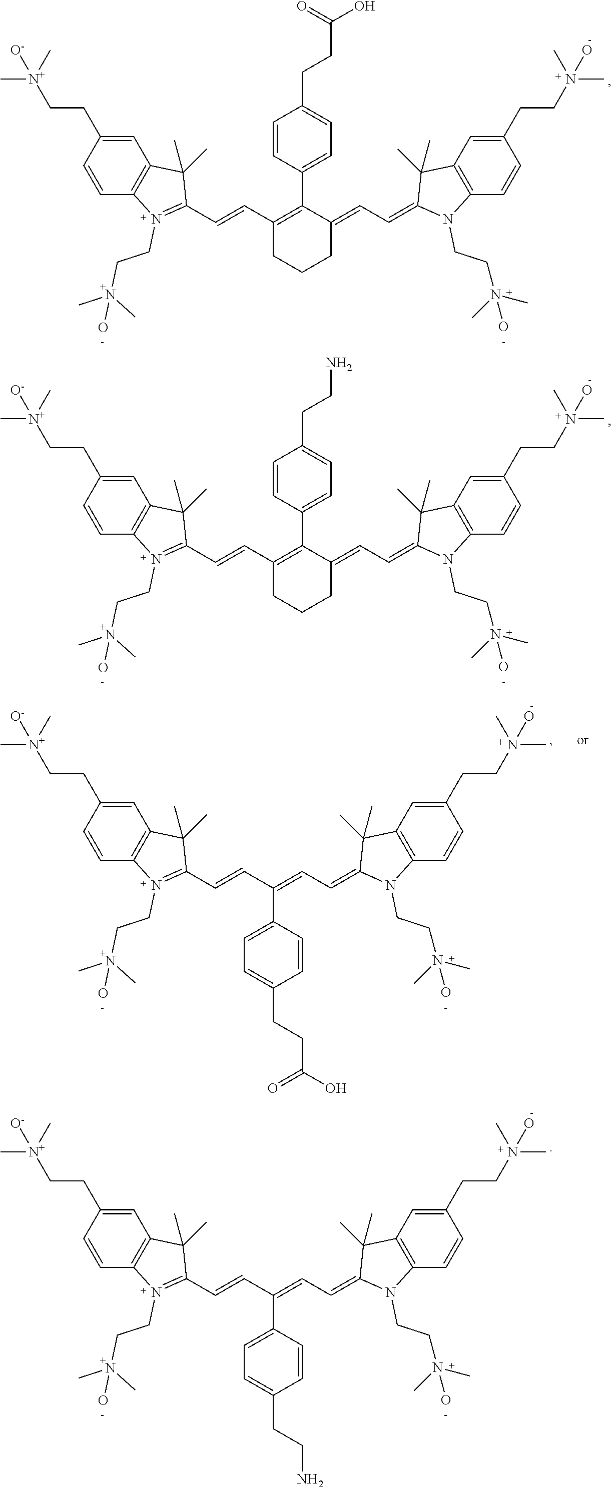

In yet another embodiment of the imaging agent dye according to the disclosure, the imaging agent is:

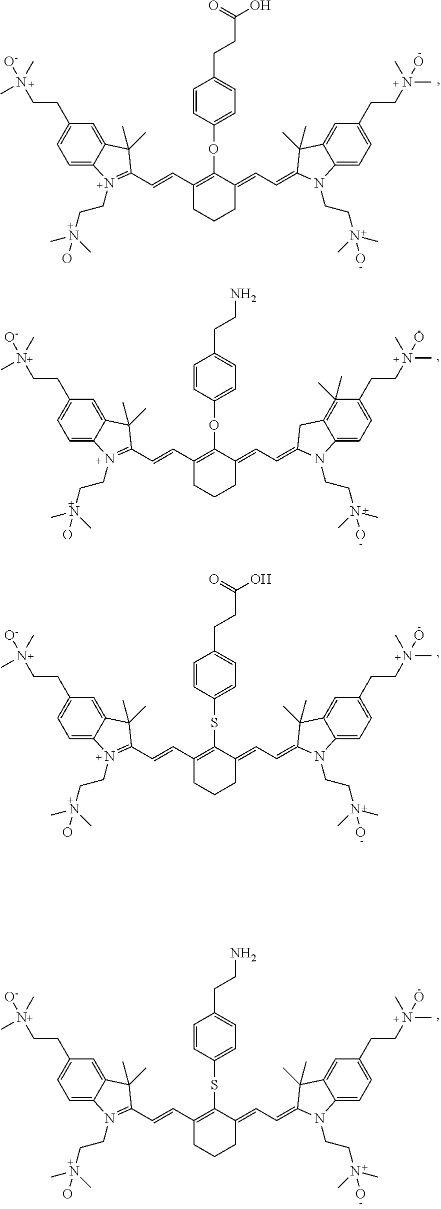

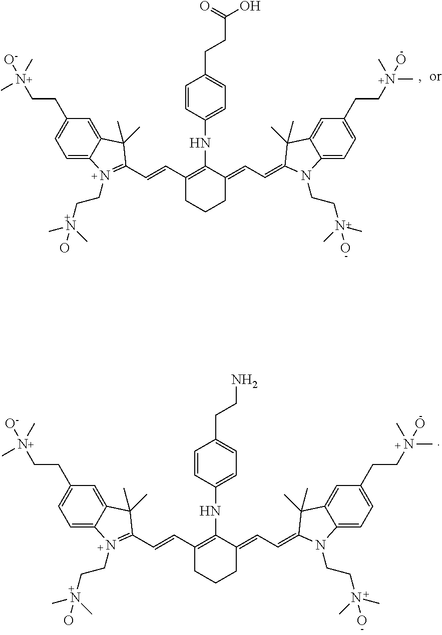

In certain embodiments of the imaging agent dye according to the disclosure, the targeting vector is cRGD, dPSMA-617, KUE, a FAP binding vector, octreotide, bombesin, or a GPI targeting vector.

In other embodiments of the imaging agent dye according to the disclosure, the imaging agent is conjugated to the targeting vector via a direct bond, or via a linking group.

In another aspect, the disclosure provides for a method of imaging tissue, cells, or lumen in a subject, the method comprising:
(a) contacting the tissue, cells or lumen with an imaging agent comprising a dye comprising an imaging agent according to the disclosure to the subject,
(b) irradiating the tissue, cells, or lumen at a wavelength absorbed by the dye;
(c) and detecting a signal from the imaging agent, thereby imaging the tissue, cells, or lumen.

In certain embodiments of the method of imaging tissue, cells, or lumen in a subject, the cells are tumor cells. In certain embodiments, the subject is human.

In some embodiments of the method of imaging tissue, cells, or lumen in a subject, the imaging agent has peak absorbance at about 600 nm to 850 nm.

In other embodiments of the method of imaging tissue, cells, or lumen in a subject, the tissue or cells is imaged in vivo.

In certain embodiments of the method of imaging tissue, cells, or lumen in a subject, the imaging agent further comprises a PEG-moiety.

In other embodiments of the method of imaging tissue, cells, or lumen in a subject, the imaging agent further comprises a radioisotope for either single-photon emission computed tomography (SPECT) or positron emission tomography (PET).

In still other embodiments of the method of imaging tissue, cells, or lumen in a subject, the imaging agent comprises a reactive linking group, such as NHS ester, sulfo-NHS ester, or a TFP ester.

In yet another aspect, the disclosure provides a method of treating cancer in a subject, the method comprising:
(a) administering an imaging effective amount of an imaging agent according to the disclosure to a subject,
(b) irradiating the cells, tissues or organs of a subject suspected of being cancerous at a wavelength absorbed by the imaging agent;
(c) diagnosing the cancer in the cells tissues, or organs of the subject by detecting a signal from the imaging agent; and
(d) administering a chemotherapeutic treatment, a radiotherapeutic treatment, or a surgical treatment to the subject to treat the cancer.

Other features and advantages of the invention will be apparent from the following detailed description, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 a schematic overview of the structure of N-oxide zwitterionic NIR fluorophores of the invention.

FIG. 2 is a representation of four specific N-Oxide zwitterionic NIR fluorophores of the invention.

FIG. 3. is a representation of a synthetic scheme outlining the first three stages of synthesis of N-Oxide zwitterionic NIR fluorophores of the invention.

FIG. 4. is a representation of a synthetic scheme outlining the condensation stage of synthesis of N-Oxide zwitterionic NIR fluorophores of the invention. The scheme shows two different condensation reactions to provide different resonant cores to the fluorophores.

FIG. 5. is a representation of a synthetic scheme outlining an SN1 reaction scheme for N-Oxide zwitterionic NIR fluorophores of the invention—having a C—O or a C—S linkage.

FIG. 6. is a representation of a synthetic scheme outlining a Suzuki coupling reaction scheme for two different N-Oxide zwitterionic NIR fluorophores of the invention—having a C—C linkage.

FIG. 7 is a representation of eight additional N-Oxide zwitterionic NIR fluorophores of the invention.

FIG. 8 is a representation of a synthetic scheme outlining additional synthetic paths for heptamethine-type N-Oxide zwitterionic NIR fluorophores of the invention.

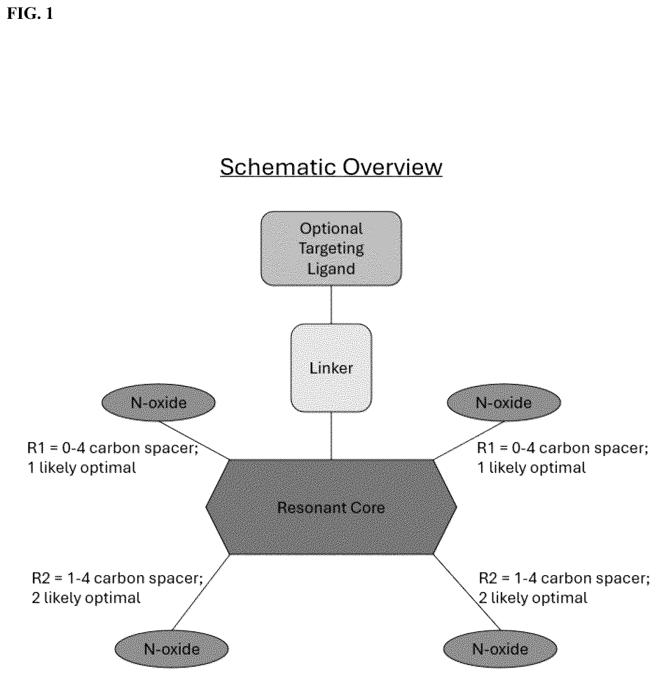

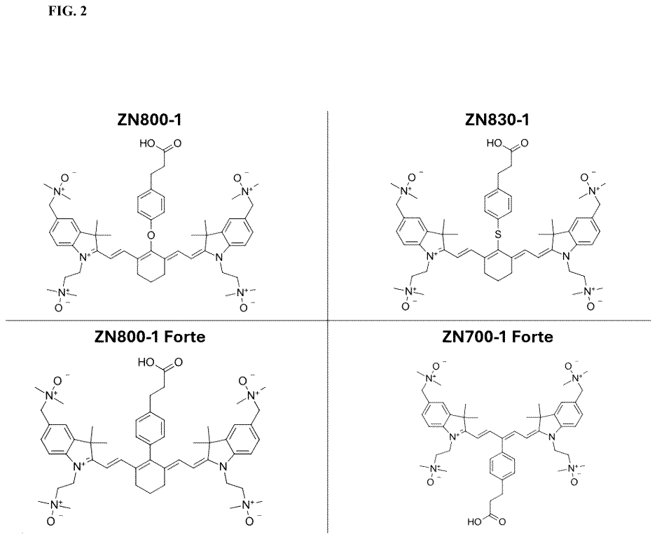

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

The present disclosure relates, inter alia, to an imaging agent that is composed of a dye molecule optionally conjugated to a targeting ligand through a linking group. The imaging agent described herein is useful in, for example, the detection of abnormal or diseased biological tissues and cells. The conjugate is particularly useful for imaging whole organisms, because it has improved in vivo behavior, such as low non-specific binding to non-targeted tissues and ultrahigh stability, resulting in an improved target-to-background ratio in connection with the detected optical signal. It is believed that these improved in vivo properties result from the balancing of formal charges on the conjugate, rendering a “charge-balanced” molecule having a net charge that is neutral or close to neutral.

### Definitions and Additional Embodiments

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

The following definitions will be useful in understanding the instant invention.

In some embodiments, imaging agents of the invention can also comprise a targeting vector for an agricultural process, chemical process, disease, or tissue-specific epitope, such as the cyclic peptide cRGDyK (aka cRGD) bound to the imaging agent. cRGD is a cyclic derivative of the tripeptide Arg-Gly-Asp which can be conjugated to one or more of the imaging agents of the invention. In still other embodiments, the targeting vector is octreotide or bombesin. In other embodiments, the targeting vector is KUE or dPSMA-617, a small molecule capable of targeting Fibroblast Activation Protein (FAP) also called FAP-inhibitor or FAPI, an amino acid or combination of amino acids, or derivatives thereof. In such embodiments, the targeting vector-conjugates can be formed in place of one or more zwitterionic groups. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

An ideal zwitterionic imaging dye conjugated to a targeting vector would adopt the total net charge of the targeting vector, which is purposeful because in most cases the charges on the targeting vector are crucial for the ability to bind its target. Targeted zwitterionic imaging dyes thus retain the major advantage of minimizing non-specific binding while maximizing specific binding. It should be apparent to those skilled in the art that additional charges can be added to the zwitterionic imaging dyes, if needed, to balance overall surface charge to zero.

In certain embodiments, the imaging dye can include a reactive linking group. Such reactive linking groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a compound of the invention and/or prodrugs thereof to a subject in need of diagnosis or treatment.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

As used herein, the term “contacting” refers to the bringing together of substances in physical contact such that the substances can interact with each other. For example, when an imaging agent is “contacted” with tissue or cells, the tissue or cells can interact with the imaging agent, for example, allowing the possibility of binding interactions between the agent and molecular components of the tissue or cells. “Contacting” is meant to include the administration of a substance such as an imaging agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO31−), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR3+), where R is independently selected from C1-6 alkyl, aryl, and arylalkyl. Other charged cationic substituents include protonated primary, secondary, and tertiary amines, and well as guanidinium. In some embodiments, the charged substituent is N(CH3)3+.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, a ether group, a sulfide group, an amino group, an alkylene group, an amide group, a carbonyl group, or a phosphate group.

Some examples of solubilizing groups include polyethylene glycols, such as (CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 250. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂)_{c}—(OCH₂CH₂)_{d}—ORa, wherein “c” is 0 to 6, “d” is 1 to 200, and Ra is H or C1-6 alkyl. In some embodiments, “c” is 1 to 4, “d” is 1 to 10, and Ra is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −1 are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a −1 charge. Suitable counterions with −1 charge include F—, Cl—, Br—, I—, sulfate, phosphate, perchlorate, acetate, trifluoroacetate, maleate, fumarate, mesylate, lactate, pyruvate, laevulinate, gluconate and the like.

### Imaging Dyes

In at least one aspect, the invention provides an imaging agent dye comprising an imaging agent optionally conjugated to targeting vector. In certain embodiments, the imaging agent can have one or more N-oxide based zwitterionic groups, which can have the formulas shown herein and in the Figures. The imaging agents of the invention improve solubility and minimize non-specific interactions. It is believed that these improved properties result from the balancing of formal charges on imaging agent, rendering a polyionic yet “charge-balanced” molecule having a net charge that is neutral or close to neutral, with an extended sphere of hydration.

The imaging agent of the invention is particularly advantageous because their behavior in vivo is believed to contribute to superior optical imaging properties and superior stability. More specifically, the charge-balancing is believed to impart good biodistribution and clearance properties to the agents, and reduce undesirable non-specific binding while the inclusion of the targeting ligand increases the time of circulation and contact time with the target. These in vivo properties help improve the target-to-background ratio of imaged tissues, leading to higher resolution imaging.

In at least one aspect, the invention provides, an imaging agent can include, a dye having one or more N-oxide based zwitterionic groups. In accordance with at least one aspect of the invention, imaging dye can be conjugated to a targeting ligand or targeting vector, discussed in the following section.

In at least one aspect, the imaging agent utilized in the invention is an agent of formula (I):

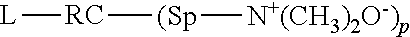

wherein for Formula (I):
L represents a linking group capable of conjugating to a targeting vector;
RC represents a resonant core;
Each Sp independently represents a spacer group; and
p represents an integer from 1-4.

In at least one aspect, the imaging agent utilized in the invention is an agent of formula (II):

### (II):

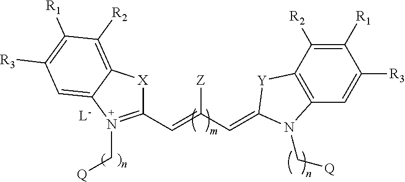

wherein for Formula (II):
each R₁ is independently —C₁-C₄ alkyl-N⁺(CH₃)₂O⁻;
each R₂ is independently H, OR′, halogen, sulfonate, substituted or unsubstituted amino, C(O)NH—C₁-C₆ alkyl, C₁-C₆ alkyl, C₁-C₆ alkoxy or phenyl;
each R₃ is independently H, OR′, halogen, sulfonate, substituted or unsubstituted amino, C(O)NH—C₁-C₆ alkyl, C₁-C₆ alkyl, C₁-C₆ alkoxy or phenyl;
   or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
   or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₂)₂O⁻;
X and Y are each independently O, S, Se, C(R″)₂, NR′″;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
each R′ is independently H, alkyl or aryl;
each R″ is independently H or alkyl;
each R′″ is independently H, akyl, akyl-SO₃H, or akyl-COOH;
m is an integer from 0-3; and
each n is independently an integer from 1-4; and
L is an anion;
or a salt, solvate, hydrate, polymorph, prodrug, or stereoisomer thereof,
and wherein the Z group is capable of conjugating a targeting vector.

In at least one aspect, the imaging agent utilized in the invention is an agent of formula (III):

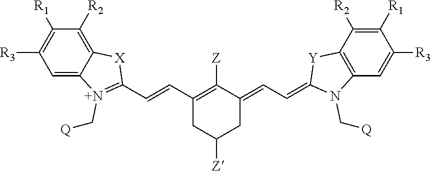

wherein for Formula (III):
each R₁ is independently —C₁-C₄ alkyl-N⁺(CH₃)₂O⁻;
each R₂ is independently H, OR′, halogen, sulfonato, substituted or unsubstituted amino, C(O)NH—C₁-C₆ alkyl, C₁-C₆ alkyl, C₁-C₆ alkoxy or phenyl;
each R₃ is independently H, OR′, halogen, sulfonato, substituted or unsubstituted amino, C(O)NH—C₁-C₆ alkyl, C₁-C₆ alkyl, C₁-C₆ alkoxy or phenyl;
   or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
   or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₂)₂O⁻;
X and Y are each independently O, S, Se, C(R″)₂, NR;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
Z′ is H, or COOR;
R is independently H, OR″″,
each R′ is independently H, alkyl or aryl;
each R″ is independently H or alkyl;
each R′″ is independently H, akyl, akyl-SO₃H, or akyl-COOH;
each R″″ is independently H, akyl, or aryl, NH₂, NHR, alkyl-NH₂, or alkyl-COOH;
each n is independently an integer from 1-4; and
L is an anion;
or a salt, solvate, hydrate, polymorph, prodrug, or stereoisomer thereof;
and wherein the Z group is capable of conjugating a targeting vector.

In certain embodiments of the invention, the imaging dye according to the invention has the following structure (or is a salt, solvate, or hydrate thereof, or is a dimer thereof):

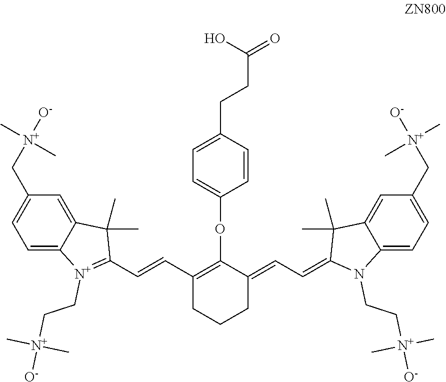

In certain embodiments of the invention, the imaging dye according to the invention has the following structure (or is a salt, solvate, or hydrate thereof, or is a dimer thereof):

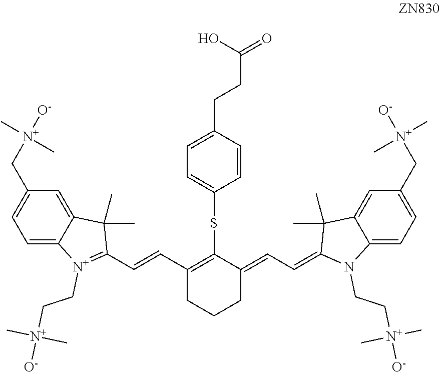

In certain embodiments of the invention, the imaging dye according to the invention has the following structure (or is a salt, solvate, or hydrate thereof, or is a dimer thereof):

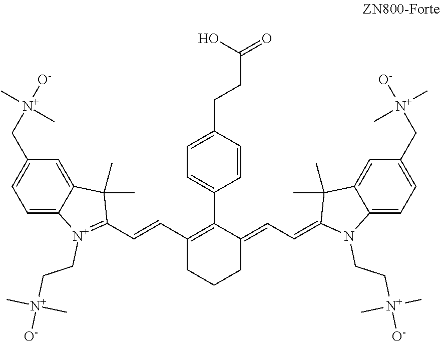

In certain embodiments of the invention, the imaging dye according to the invention has the following structure (or is a salt, solvate, or hydrate thereof, or is a dimer thereof):

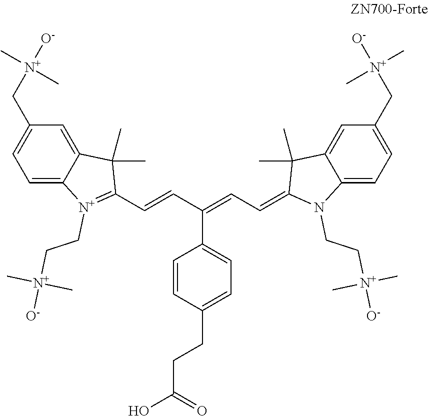

In certain embodiments of the invention, the imaging dye according to the invention has the following structure (or is a salt, solvate, or hydrate thereof, or is a dimer thereof):

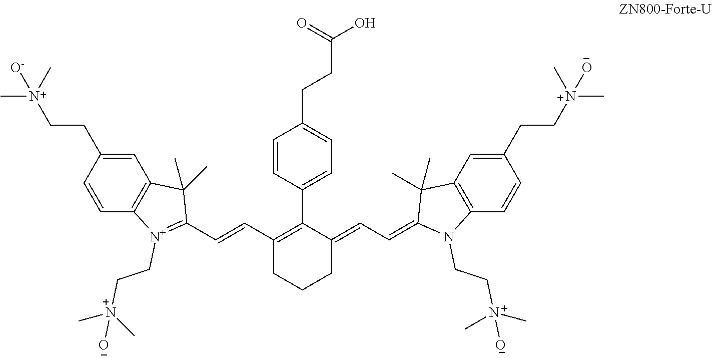

In certain embodiments of the invention, the imaging dye according to the invention has the following structure (or is a salt, solvate, or hydrate thereof, or is a dimer thereof):

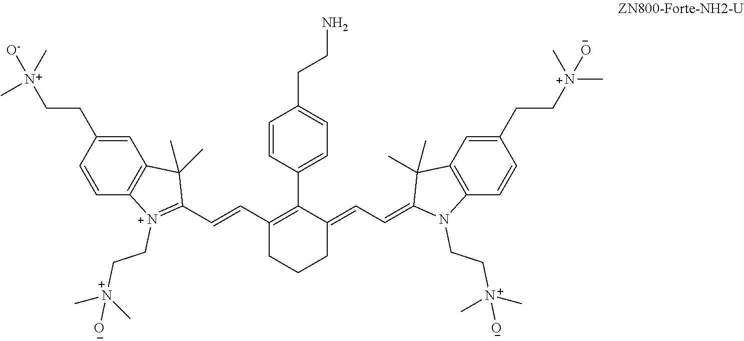

In certain embodiments of the invention, the imaging dye according to the invention has the following structure (or is a salt, solvate, or hydrate thereof, or is a dimer thereof):

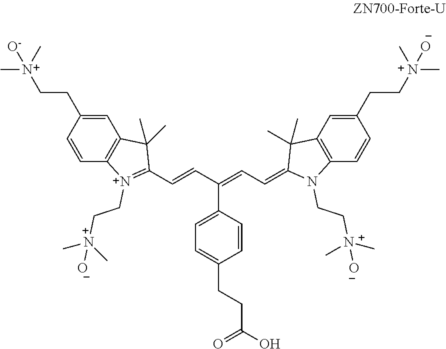

In certain embodiments of the invention, the imaging dye according to the invention has the following structure (or is a salt, solvate, or hydrate thereof, or is a dimer thereof):

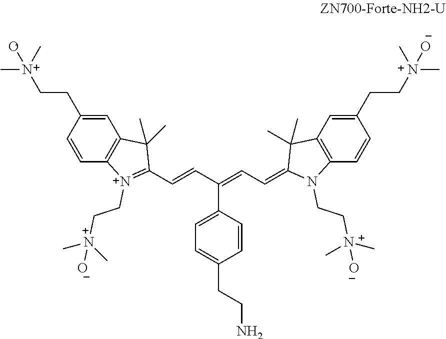

In certain embodiments of the invention, the imaging dye according to the invention has the following structure (or is a salt, solvate, or hydrate thereof, or is a dimer thereof):

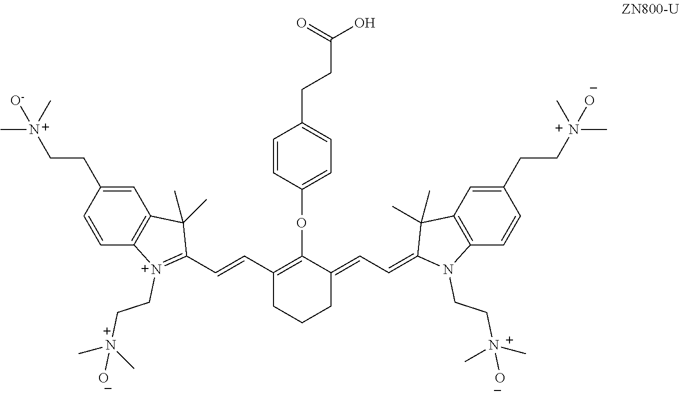

In certain embodiments of the invention, the imaging dye according to the invention has the following structure (or is a salt, solvate, or hydrate thereof, or is a dimer thereof):

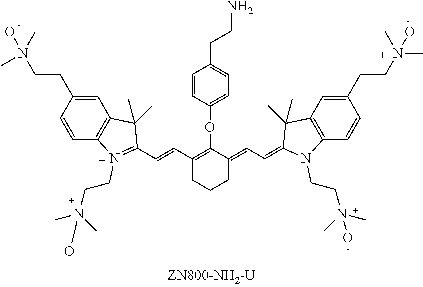

In certain embodiments of the invention, the imaging dye according to the invention has the following structure (or is a salt, solvate, or hydrate thereof, or is a dimer thereof):

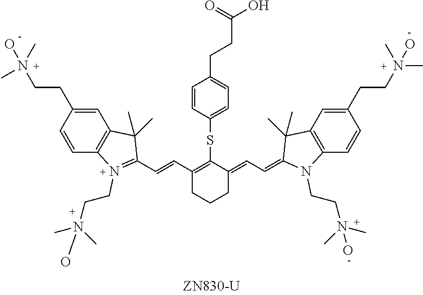

In certain embodiments of the invention, the imaging dye according to the invention has the following structure (or is a salt, solvate, or hydrate thereof, or is a dimer thereof):

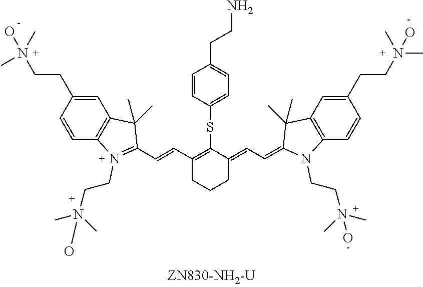

In certain embodiments of the invention, the imaging dye according to the invention has the following structure (or is a salt, solvate, or hydrate thereof, or is a dimer thereof):

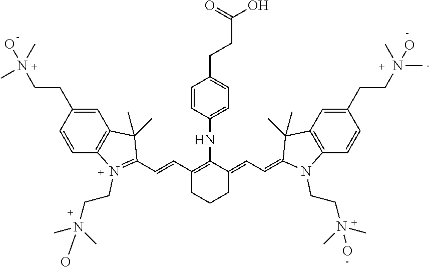

In certain embodiments of the invention, the imaging dye according to the invention has the following structure (or is a salt, solvate, or hydrate thereof, or is a dimer thereof):

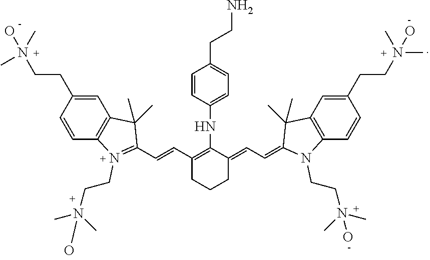

In certain embodiments of the invention, the imaging dye according to the invention may include two or more of the imaging agents described herein. In particular embodiments, the imaging dye according to the invention may include two or more of the imaging agents, wherein the imaging agent core is the same but having different targeting vectors. In still other embodiments, the imaging dye according to the invention may include two or more of the imaging agents described herein wherein the imaging agent core is the different but having the same targeting vector—specifically, without limitation, the imaging agent cores may be chosen to absorb different wavelengths of irradiation. In still other embodiments, the imaging dye according to the invention may include two or more of the imaging agents described herein wherein the imaging agent core is the different and the targeting vector is different—specifically, without limitation, the imaging agent cores may be chosen to absorb different wavelengths of irradiation while the targeting vectors can be chosen to identify different cell/tumor types.

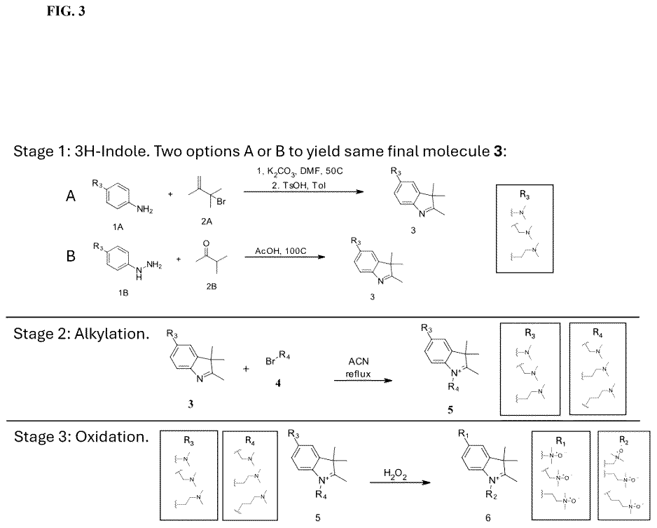

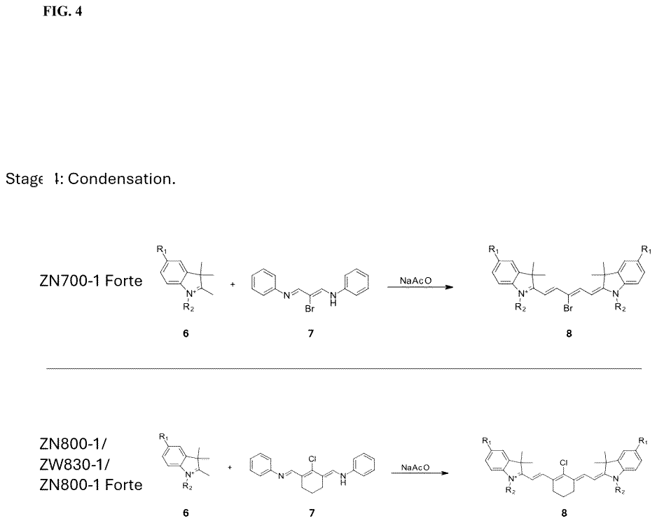

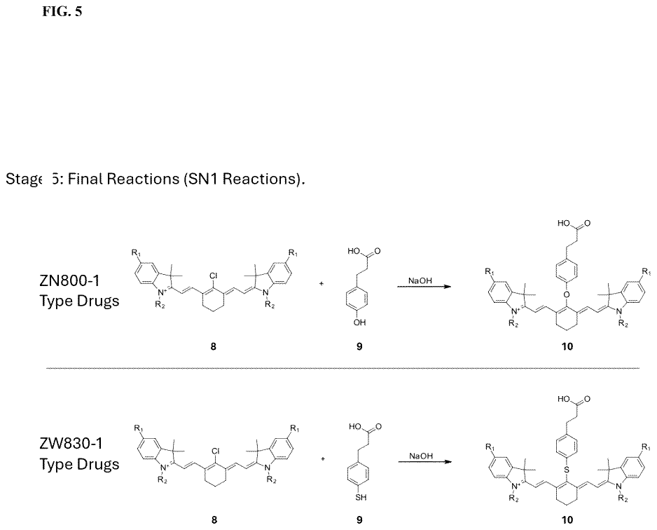

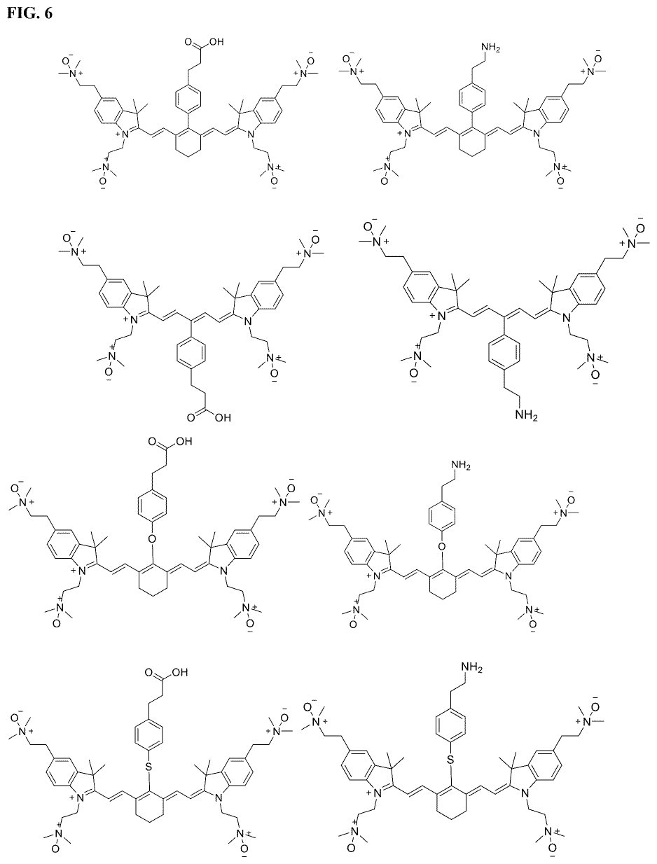

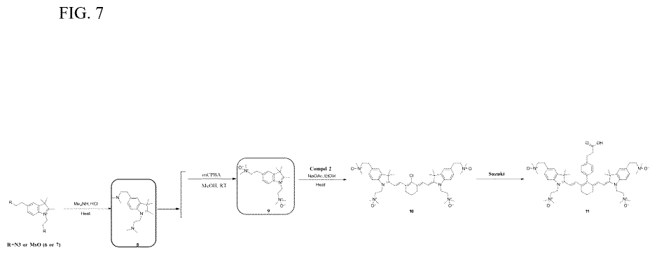

The imaging dyes of the invention can be synthesized using the protocols described in Schemes shown in FIGS. 3-6. Alternatively, N-oxide functionalities may be synthesized by nucleophilic displacement of a suitable leaving group (such as a halogenide or a sulfonate) instead of the group R in the scheme. The nucleophile in these conversions is a N,N-dialkylated hydroxylamine, which can be protected at the hydroxyl group.

### Targeting Ligands or Vectors

In at least one aspect of the invention provides, the imaging agent includes an embodiment of one of the aforementioned imaging dyes conjugated to a targeting ligand or targeting vector. In certain embodiments, the targeting vector (or targeting ligands) can be cRGD, PSMA binding vector, such as dPSMA-617 or KUE, a FAP binding vector such as NH₂-FAPI-74, a bombesin receptor binding vector, a GPI derivative, or a somatostatin receptor binding vector, or their corresponding homo- or hetero-dimers. In certain embodiments, the targeting vectors are agonists or antagonists of the target, including, but not limited to, agonists or antagonists of integrins, agonists or antagonists of FAP, agonists or antagonists of PSMA, agonists or antagonists of somatostatin receptor, and agonists or antagonists of GRPR. In still other embodiments, the targeting vector may be a derivative of the targeting vector, including, but not limited to, derivatives of cRGD, derivatives of FAP, derivatives of PSMA-binding vectors, derivatives of octreotide, and derivatives of bombesin.

In certain embodiments, the imaging agent is conjugated to the targeting vector via a direct bond, or via a linking group, for example as shown in the formulas described herein, and in the Figures. In particular embodiments, the targeting vector may be bound to the imaging agent by coordination through a free —COOH group or a free —NH₂ group in the Z or Z′ group.

In certain embodiments, one or more of the imaging agent conjugates in the combination includes cRGD as the targeting ligand.

In certain embodiments of the invention, the targeting ligand according to the invention can be a cyclic-RGD having the following structure:

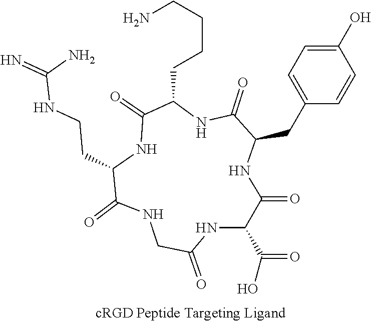 [SMILES: N=C(N)NCC[C@@H]1NC(=O)[C@H](CCCCN)NC(=O)[C@@H](Cc2ccc(O)cc2)NC(=O)[C@H](C(=O)O)NC(=O)CNC1=O]

Provided below are examples for cRGD conjugates for embodiments of the imaging agents, including cRGD-ZN800, cRGD-ZN800-Forte, cRGD-ZN830, or cRGD-ZN700-Forte. The targeting ligand can be conjugated to the imaging dye by an optional linking group or via direct bond. As shown herein, the imaging targeting ligand is conjugated to the imaging dye via an amine linkage. Nevertheless, other conjugating linkages can be used as determined by one of skill in the art. Other cRGD conjugates can be prepared without deviating from the essence of the invention.

Integrins are a family of cell adhesion receptors that provide for cell motility and invasion. The integrin family in humans comprise 18 α and β subunits which assemble into different functional heterodimers. Some integrins, act as mediators of angiogenesis in solid tumors. Integrin expression is important for tumor progression and metastasis by promoting tumor cell migration, invasion, proliferation, and survival. In addition to cancer, integrins are also highly expressed during many normal and abnormal processes, such as fibrosis, wound healing, and inflammation. Integrin αvβ3 expression has been established as a surrogate marker of angiogenic activity. Similarly, Integrin αvβ6 is a subtype of the integrin family that is expressed exclusively on epithelial cells. αvβ6 is usually expressed at low or undetectable levels in normal adult tissues but can be highly upregulated during pathological and physiological processes such as wound healing, fibrosis, inflammation, and cancer. RGD mimetics have been used in some studies as a targeting moiety to deliver integrin antagonists to a variety of cell types. However, continuous infusion of RGD peptides have been found to stimulate tumor growth (See, Reynolds, L. E. et al. Enhanced pathological angiogenesis in mice lacking β3 integrin or β3 and β5 integrins. Nature Med. 8, 27-34 (2002)). In addition, many RGD bound agents have expensive, complex, time-consuming and low-yield synthetic procedures. Similarly, with regard to imaging, degradation of agents accumulation of RGD bound agents in the bladder have been found to impair some imaging methods. Given their stability and clearance rates, the conjugates of the invention have the potential to provide a long-lasting visual identification and imaging of tumors which overexpress integrins while avoiding the setbacks of prolonged systemic administration of RGD peptide ligands.

As such, in a particular aspect, the imaging agent may be used to image or identify (for example, during surgery) integrin overproduction in the cells of a subject, for example when the imaging dye compound is conjugated to cRGD, e.g., as shown in the above structures.

In certain embodiments of the invention, the targeting ligand according to the invention can be a prostate specific membrane antigen (PSMA) binding vector. For example, dPSMA-617 or Vipivotide tetraxetan. In certain embodiments, the PSMA binding vector can be F a targeting vector based on (((S)-5-((S)-2-((1r,4S)-4-(aminomethyl)cyclohexane-1-carboxamido)-3-(naphthalen-2-yl)propanamido)-1-carboxypentyl)carbamoyl)-L-glutamic acid, which is a derivative of PSMA-617 or Vipivotide tetraxetan. This PSMA-targeting vector is called dPSMA-617 (for “derivative of PSMA-617) in this disclosured PSMA-617.

In certain embodiments of the invention, the targeting ligand according to the invention can be dPSMA-617 having the following structure:

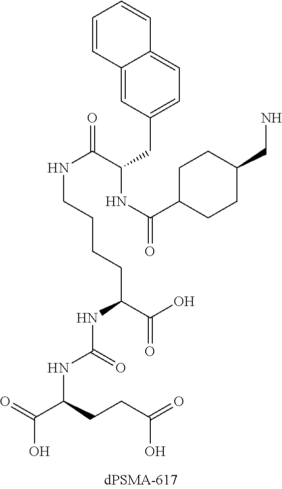

The targeting ligand can be conjugated to the imaging dye by an optional linking group or via direct bond. As shown herein, the imaging targeting ligand is conjugated to the imaging dye via an amine linkage. Nevertheless, other conjugating linkages can be used as determined by one of skill in the art. Other dPSMA-617 conjugates can be prepared without deviating from the essence of the invention.

In certain embodiments, the targeting ligand includes one or more derivatives of bombesin, including, but not limited to, BIM26226, JMV594, JMV641, JMV736, JMV717, JMV845, and JMV542. Additional bombesin derivatives, including bombesin agonists, bombesin antagonists, and bombesin pseudopeptides can be found in Azay et al., Peptides, Vol 19, No. 1, pp 57-63, 1988—which is incorporated herein by reference.

In certain embodiments of the invention, the PSMA-targeting vector is (((S)-5-amino-1-carboxypentyl)carbamoyl)-L-glutamic acid (KUE). The targeting ligand KUE can have the following structure:

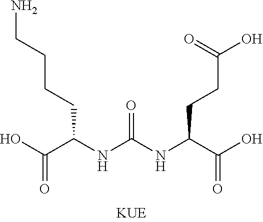 [SMILES: NCCCC[C@H](NC(=O)N[C@@H](CCC(=O)O)C(=O)O)C(=O)O]

The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other KUE conjugates can be prepared without deviating from the essence of the invention.

In certain embodiments, the FAPI-targeting ligand includes NH2-FAPI-74. Specific details regarding NH2-FAPI-74 are described in: Linder et al., “Radioligands Targeting Fibroblast Activation Protein (FAP),” the entirety of which is incorporated herein by reference.

In general, NH2-FAPI-74 has the following structure:

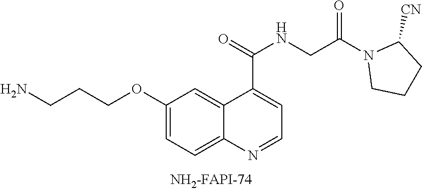 [SMILES: N#C[C@@H]1CCCN1C(=O)CNC(=O)c1ccnc2ccc(OCCCN)cc12]

The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other FAP conjugates can be prepared without deviating from the essence of the invention.

In certain embodiments of the invention, the targeting ligand according to the invention can be a bombesin receptor binding vector.

In general, the structure of bombesin (SEQ ID No.: 6) is:

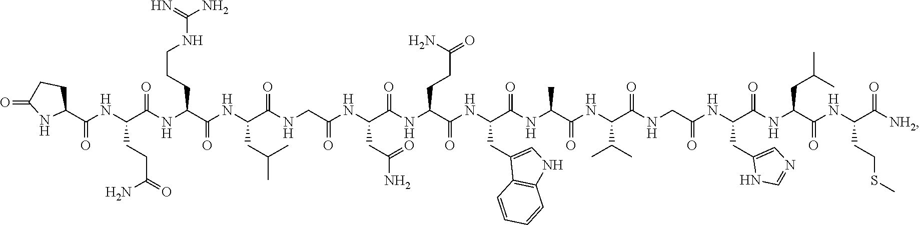 [SMILES: CSCC[C@H](NC(=O)[C@H](CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@H](CC(N)=O)NC(=O)CNC(=O)[C@H](CC(C)C)NC(=O)[C@H](CCCNC(=N)N)NC(=O)[C@H](CCC(N)=O)NC(=O)[C@@H]1CCC(=O)N1)C(C)C)C(N)=O]

or a derivative thereof.

The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other bombesin conjugates can be prepared without deviating from the essence of the invention.

In certain embodiments of the invention, the targeting ligand according to the invention can be a somatostatin receptor binding vector. In certain embodiments, the somatostatin receptor binding vector can be octreotide.

In general, the structure of octreotide (SEQ ID No.: 5) is:

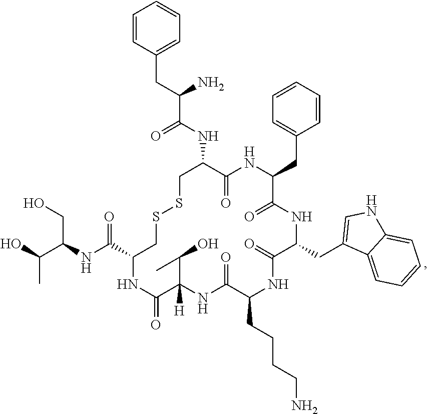 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

or a derivative thereof.

The targeting ligand can be conjugated to the imaging dye by an optional linking group or via direct bond. As shown herein, the imaging targeting ligand is conjugated to the imaging dye via linking group (L). Nevertheless, other conjugating linkages can be used as determined by one of skill in the art. Other OCTREOTIDE conjugates can be prepared without deviating from the essence of the invention.

In certain embodiments, the targeting ligand includes a phosphinate mimic of N-acetylaspartylglutamic acid (GPI) or a derivative thereof. Particular derivatives of GPI can be found in U.S. Provisional Ser. No. 63/858,228 filed Aug. 5, 2025.

In certain embodiments, the GPI targeting ligand may serve as the pharmacophore component that provides selective binding to prostate-specific membrane antigen (PSMA). The GPI targeting ligand may comprise a glutamate portion linked to a phosphoramidate portion, forming a bifunctional binding unit that engages multiple sites within the PSMA active site.

In certain embodiments, the GPI targeting ligand comprises a general chemical structure, where n=0 to 3 for Regions A and D:

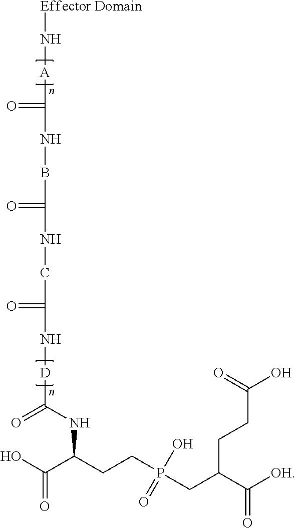

Three key examples of GPI targeting ligand includes:

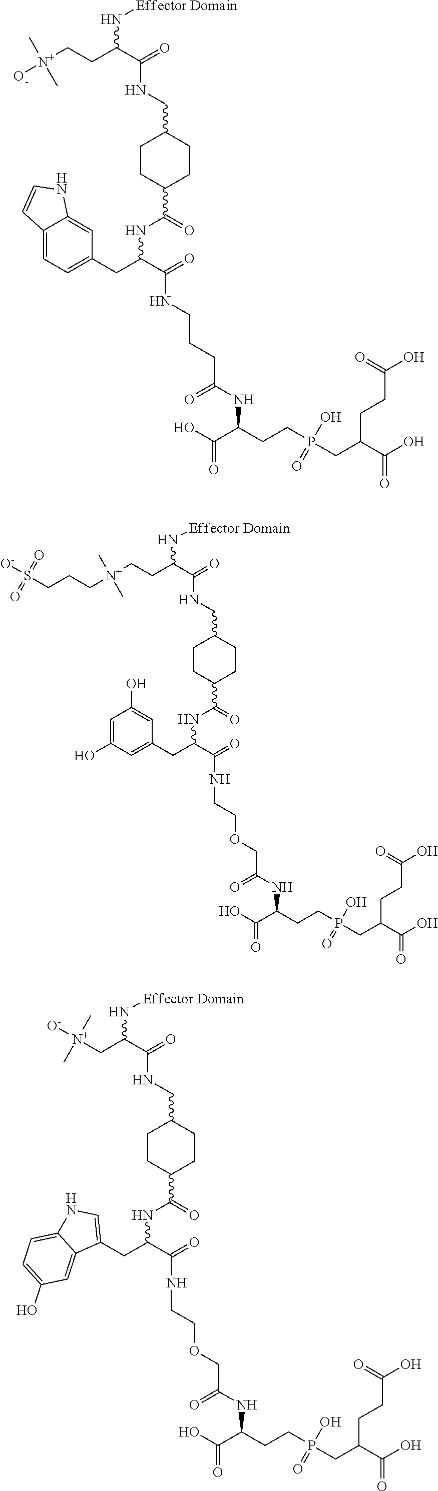

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GEl 1, a dodecapeptide, binding to epidermal growth factor receptor (EGFR or ErbB1) for diagnosing/treating tumors of epithelial origin.

Accordingly, the invention provides an imaging agent dye comprising an imaging agent conjugated to a targeting vector. In certain embodiments of the imaging agent dye according to the invention, the imaging agent can be ZN800, ZN830, ZN800-Forte, ZN700-Forte, ZN800-U, ZN830-U, ZN800-U or ZN700-U, ZN800-NH₂—U, ZN830-NH₂—U, ZN800-NH₂—U or ZN700-Forte-NH₂—U, and the targeting vector can be a PSMA ligand, for example, KUE or dPSMA-617, a FAP ligand, a bombesin receptor ligand, or a somatostatin receptor ligand, as shown and described. The imaging agent can be conjugated to the targeting vector via a direct bond, or via a linking group.

Any suitable combination of imaging agent and targeting ligand can be used, for example, the imaging agents discussed herein, e.g., ZN800, ZN830, ZN800-Forte, ZN700-Forte, ZN800-U, ZN830-U, ZN800-U or ZN700-U, ZN800-NH₂—U, ZN830-NH₂—U, ZN800-NH₂—U or ZN700-Forte-NH₂—U can be used with any one of the targeting vectors discussed herein, e.g., cRGD, PSMA binding vector, such as dPSMA-617 or KUE, a FAP binding vector, a bombesin receptor binding vector, or a somatostatin receptor binding vector.

In each combination, the respective targeting ligand or vector can be covalently attached to a respective reactive linking group of a respective dye compound of the invention through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

In certain embodiments, the targeting vector can be a targeting ligand and can be covalently attached to a reactive group of the dye compound, or through an optional linking group (L), through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

As used herein, “linking group” refers to any molecular entity having a molecular weight from about 50 to about 500 Da that is capable of conjugating with a targeting ligand (TL). In particular, the linking group includes at least one reactive group selected from a carboxylic acid group or anhydride or ester thereof, as well as an isothiocyanate group. In some embodiments, the linking group contains a carboxylic acid group. In some embodiments, the linking group is a PEG moiety or a straight or branched chain hydrocarbon moiety having between 2 and 12 carbon atoms. In some embodiments, the linking group includes a branching point, a reactive linking group, or other reactive group which can be used to further functionalize the imaging agent, for example, for inclusion of a radioisotope.

In certain embodiments, the targeting ligand can further include a molecular scaffold moiety to which the binding moiety and other groups can attach. For example, the molecule scaffold can bear one or more of the following: (1) a moiety designed to react with the reactive linking group of the dye to form a covalent bond, (2) a charge balancing moiety, such as any of the ionic groups described herein, and (3) a moiety that binds to the biological target. An example of a molecular scaffold is an adamantane derivative, such as described in U.S. Pat. App. Pub. No. 2006/0063834, which is incorporated herein by reference in its entirety, and illustrates the preparation of a targeting ligand that incorporates an adamantane scaffold. Specifically, the adamantane core holds (1) an amino group capable of reacting with the dye compounds, (2) a charge-balancing moiety that will neutralize a negative charge on the dye molecule, and (3) two moieties that bind to the biological target PSMA. For a description of moieties that bind to PSMA, see, Humblet, V. et al. Mol. Imaging, 2005, 4: 448-62; Misra P. et al. J. Nucl. Med. 2007, 48: 1379-89; Chen, Y., et al. J. Med. Chem, 2008, 51: 7933-43; Chandran, S. S., et al. Cancer Biol. Ther., 2008, 7:974-82; Banerjee, S. R., J. Med. Chem. 2008, 51: 4504-17; Mease, R. C., et al. Clin. Cancer Res., 2008, 14:3036-43; Foss, C. A. et al. Clin. Cancer. Res., 2005, 11:4022-8, each of which is incorporated herein by reference in its entirety.

In certain embodiments, ZN800, ZN830, ZN800-Forte, ZN700-Forte, ZN800-U, ZN830-U, ZN800-U or ZN700-U, ZN800-NH₂—U, ZN830-NH₂—U, ZN800-NH₂—U or ZN700-Forte-NH₂—U, targeting ligands, and imaging agents can be isolated as salts, acids, bases, or combinations thereof. For example, dyes, conjugates, and imaging agents having multiple charged substituents can be isolated by introducing counterions and/or protons sufficient to counter the charges of the various substituents normally present in neutral pH so that the dye, conjugate, or imaging agent can be isolated, for example, as a solid substance.

In certain embodiments, the imaging agent further comprises a PEG-moiety. Such moiety can be bound to the conjugate at any suitable structural location as would be understood by one of ordinary skill in the synthesis of such compounds. In certain embodiments, the agent further comprises a PEG-moiety to alter the circulation time in blood. Such moiety can be bound to the conjugate at any suitable structural location as would be understood by one of ordinary skill in the synthesis of such compounds. In addition, in certain embodiments, the PEG-moiety can be included as the optional linking group between ZN800, ZN830, ZN800-Forte, ZN700-Forte, ZN800-U, ZN830-U, ZN800-U or ZN700-U, ZN800-NH₂—U, ZN830-NH₂—U, ZN800-NH₂—U or ZN700-Forte-NH₂—U, and the targeting ligand using methods known to one of ordinary skill in the art.

In certain embodiments, the imaging agent can include a radioisotope for either single-photon emission computed tomography (SPECT) or positron emission tomography (PET). Such radioisotope can be bound or further conjugated to the conjugate at any suitable structural location as would be understood by one of ordinary skill in the synthesis of such compounds.

In certain embodiments, the imaging agent can comprise a reactive linking group. Such reactive linking groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

### Imaging Methods, Methods of Identifying and Imaging Tumors, and Methods of Treating Cancer

Embodiments of the imaging agents, including dye compounds conjugated to antigen specific targeting vectors can be particularly advantageous because their behavior in vivo can contribute to superior optical imaging properties, for example, by significantly increasing the target-to-background ratio of imaged tissues, leading to higher resolution imaging, and ultimately providing for better recognition of malignant tissue for resection and margin assessment and improved visualization during minimal invasive laparoscopic surgery, for example. Performing complete and accurate resections of cancerous tissues during surgery is critical, thus higher target-to-background ratios observed by using embodiments of the imaging agents in conjunction with one or more embodiments of the inventive methods described herein, provides clinicians with greater visualization to ensure resections are complete.

Moreover, embodiments of imaging agents according to the invention include a labile linking bond that when broken renders the fluorophore non-fluorescent in the NIR spectrum and separates the targeting ligand from the fluorophore. If the targeting ligand binds to a cell and is internalized within lysosomes or other acidic intracellular compartments, the imaging dye is stabilized. The NIR fluorophores of this invention are highly stable in acidic environments and thus display maximum NIR fluorescence. On the contrary, non-bound targeted fluorophore is degraded over time in blood, thus lowering the background significantly. The combination of increased NIR fluorophore stability after targeting and internalization and decreased stability in blood results in a counterintuitive result that forms the basis of this invention.

In at least one aspect, the invention provides, a method of imaging cells, e.g., tumor cells in a subject, where the cells can be tumor cells, inflammatory cells, or cells undergoing angiogenesis, and the subject can be a human subject, and further, the cells can be imaged in vivo.

The methods of imaging tissue, cells, or lumen include the following basic steps:
(a) contacting the tissue, cells, or lumen with an imaging agent comprising a conjugate of a dye, the conjugate comprising a PSMA, FAP, bombesin receptor, or somatatostatin receptor targeting ligand bound to the dye;
(b) irradiating the tissue or cells at a wavelength absorbed by the conjugate; and
(c) detecting an optical signal from the irradiated tissue or cells.

In certain embodiments, the method according to the invention can include the following steps:
a) administering an imaging effective amount of an imaging agent according to any one or more embodiments of the invention described herein to the subject;
b) 2-4 hours after administration of the imaging agent, irradiating a region in the subject in which tumor cells are expected to be found with at a wavelength absorbed by the imaging agent; and
c) detecting a signal from the imaging agent.

In at least one aspect, the invention provides a method of imaging cells, and further, a method of treating cancer in a subject.

In certain embodiments of the method according to the invention, the method of treating cancer can further include, imaging the cells, which can include the following steps:
a) administering an imaging effective amount of an imaging agent according to any one or more embodiments of the invention described herein (e.g., an imaging agent comprising a conjugate of a dye, the conjugate comprising a PSMA, FAP, bombesin receptor, or somatostatin receptor targeting ligand bound to the dye) to a subject; and
b) irradiating the cells, tissues or organs of a subject suspected of being cancerous at a wavelength absorbed by the imaging agent. In certain embodiments, the imaging agent has peak absorbance at about 600 nm to 850 nm.
   The method for treating cancer, can further include the following steps,
c) diagnosing the cancer in the cells tissues, or organs of the subject by detecting a signal from the imaging agent; and
d) administering a chemotherapeutic treatment, a radiotherapeutic treatment, or a surgical treatment to the subject to treat the cancer.

In certain embodiments, the method is suitable for imaging of abnormal, but not malignant, tissue, such as defects of the musculoskeletal system using FAP as a targeting ligand, vascular system using cRGD as a targeting ligand, melanoma using LyP-1 peptide as a targeting ligand, breast cancer using K237 peptide and/or mUNO as a targeting ligand, Lung tumor, breast tumor, colon tumor using IL4RPep-1 as a targeting ligand.

In certain embodiments, the method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain/cns cancer (e.g., gliomas), throat cancer, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

In certain embodiments, the cancer cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In particular embodiments, the cancer cells are prostate cancer cells.

In certain embodiments, the biological sample is part or all of a subject. In some embodiments, the biological sample is obtained from a subject.

In particular embodiments, the method includes the steps of (a) contacting the biological sample with a combination of the imaging agent conjugates described above, wherein each of the imaging agent conjugates in the combination comprises a targeting ligand linked to an imaging agent. The imaging agent is capable of being detected by one or more conventional scanning methods.

In certain embodiments, the compound is administered by parenteral, intranasal, sublingual, rectal, or transdermal delivery. In some such embodiments, the compound is administered intravenously. In some embodiments, the compound is administered intratumorally.

In particular embodiments, the cells are tumor cells. For example, and without limitation, the tumor cells are melanoma cells, sarcoma cells, musculoskeletal tumor cells, breast cancer tumor cells, renal cancer cells, rectal cancer tumor cells, bone metastases, neuroendocrine tumor cells, brain metastases, glioblastoma multiforme (GBM) cells, squamous cell carcinoma cells of the head and neck (SCCHN), or non-small cell lung cancer (NSCLC) cells. As such, the invention provides for a method of imaging such tumors in a subject.

In certain embodiments, the cells are associated with inflammation or angiogenesis. For example, and without limitation, the cells may be associated with inflammatory lesions, endometriosis, ischemic injury, cardiovascular disease, neurovascular disease, myocardial infarction, moyamoya disease, stroke, atherosclerosis, or rheumatoid arthritis.

In certain embodiments, and in at least one aspect, the invention provides, the method of imaging cells includes, contacting cells with an embodiment of an imaging agent according to any one or more embodiments of the invention described herein (e.g., having one of the formulas shown and described herein), irradiating the cells at a wavelength absorbed by the imaging agent, and detecting a signal from the imaging agent, thereby imaging the cells. The imaged cells can be tumor cells, inflammatory cells, or cells undergoing angiogenesis.

In certain embodiments of the method of imaging cells according to the invention, an embodiment of the imaging agent is administered to an organism comprising or suspected of comprising the cells. The imaging agent described herein is a substance that can be used to image tissues or cells, such as those of a living organism, for purposes of diagnosis, therapy, image-guided surgery, and the like. In some embodiments, the organism is a mammal, such as a human.

Certain embodiments of the imaging agent of the invention contains a dye that is capable of absorption of electromagnetic radiation, typically in the ultraviolet (UV), visible, or near infrared (NIR) range. The imaging agent can also be capable fluorescent emission, such as in the visible or NIR range. The optical signal detected from the dye or conjugate can be, for example, absorption or fluorescent emission. In some embodiments, fluorescent emission from the dye is the primary optical signal detected for imaging purposes. In some embodiments, the dye has a peak absorbance at about 525 nm to about 850 nm, at about 550 nm to about 825 nm, about 600 nm to about 825 nm, about 700 nm to about 825 nm, or at about 750 nm to about 825 nm. In some embodiments, the dye has a peak fluorescent emission at about 700 nm to 875 nm, about 725 to about 850 nm, about 750 to about 850 nm, or about 775 to about 850 nm. In particular embodiments, the conjugated imaging agent of the invention has a peak fluorescent emission at about 700 nm.

Certain embodiments of the imaging agent is generally “charge-balanced,” unless otherwise specified, which refers to having a net overall charge of zero, or close to zero, such as +1 or −1. Charge-balancing occurs when negatively charged substituents on the imaging agent are countered by the presence of an equal number, or close to an equal number, of positively charged substituents on the same molecule, and vice versa. In some embodiment, the net charge is 0 or +1. In some embodiments, the net charge is 0. In some embodiments, the net charge is +1. In further embodiments, the net charge is −1. The value “n” in the formulae provided herein represents net charge.

Certain embodiments of the imaging agent described herein generally has improved “target-to-background ratio” (TBR) compared to presently known fluorescent imaging agents. The improvement in TBR is believed to be a result of improved in vivo properties due to “charge-balancing.” TBR is a measure of the intensity of the fluorescent signal obtained from a target (peak signal) over the measure of the intensity of the fluorescent signal obtained nearby the target (background signal), the target being the tissues or cells targeted by the imaging agent. TBR measurements can be readily obtained through routine measurement procedures. For fluorescent imaging systems, and other optical-type systems, digital images recording optical signals of the target facilitate TBR measurement. Higher TBR values are more desirable, resulting in greater resolution of the imaged tissues. In some embodiments, the imaging agents achieve an TBR of at least about 1.1 (i.e., peak signal is at least 10% over background).

Certain embodiments of the imaging agent of the invention generally includes one or more ionic groups. In some embodiments, the imaging agents include two or more, three or more, four or more, or five or more ionic groups. Ionic groups serve to increase solubility of the generally hydrophobic dye portions of the imaging agent, thus, improving biodistribution. The ionic groups can be located on any portion of the imaging agent, such as the dye portion, the targeting ligand, or both.

The term “ionic group” refers to a moiety comprising one or more charged substituents. The “charged substituent” is a functional group that is generally anionic or cationic in substantially neutral aqueous conditions (e.g. a pH of about 6.5 to 8.0, or preferably about physiological pH (7.4)). Examples of charged anionic substituents include anions of inorganic and organic acids such as sulfonate (—SO₃¹⁻), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR₃⁺), where R is independently selected from C₁₋₆ alkyl, aryl, and arylalkyl. Other charged cationic substituents include protonated primary, secondary, and tertiary amines, and well as guanidinium. In some embodiments, the charged substituent is —N(CH₃)₃⁺. Further examples of ionic groups are described infra.

Certain embodiments of the imaging agent of the invention generally has good solubility in substantially neutral aqueous media, and in particular, blood and blood serum. In some embodiments, the imaging agent has a solubility in 10 mM HEPES solution, pH 7.4, of at least about 10 μM. In further embodiments, the imaging agent has a solubility in 10 mM HEPES solution, pH 7.4, of at least about 15 μM at least about 20 μM, at least about 25 μM, at least about 30 μM, at least about 40 μM, or at least about 50 μM.

Certain embodiments of the imaging agent of the invention generally can be neutral molecules or salts. For example, if the dye or dye conjugate is charged, the imaging agent can be or contain a salt or acid (or combination thereof) of the dye or dye conjugate. For positively charged dyes or conjugates, suitable counter ions include anions such as fluoride, chloride, bromide, iodide, acetate, perchlorate, PF₆⁻, and the like. For negatively charged dyes or conjugates, suitable counterions include cations such as Na⁺, K⁺, and quaternary amines.

Certain embodiments of the imaging agent of the invention has significant improvements with regard to stability over time, allowing for dramatically improved operability and use for imaging and mapping. Similarly, the stability of the imaging agent allows for increased accuracy during surgery as the signal does not degrade over time.

The imaging agent of the invention is particularly advantageous because their behavior in vivo is believed to contribute to superior optical imaging properties. More specifically, the charge-balancing is believed to impart good biodistribution and clearance properties to the agents, and reduce undesirable non-specific binding. These in vivo properties help improve the target-to-background ratio of imaged cells and tissues, leading to higher resolution imaging. Furthermore, the imaging agents of the inventions are cleared primarily or exclusively by the kidneys.

In some embodiments, the tumor or cell is found in a subject. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

### Dosage Forms and Administration Methods

In the methods of the invention, embodiments of the imaging agent can be administered at a predetermined dosage. The predetermined dosage amount is not particularly limited provided that the predetermined dosage is administered at least a minimum amount capable of being cleared by the kidneys within twelve hours.

In some embodiments, a detectably effective amount of the imaging agent of the presently disclosed methods is administered to a subject. In accordance with the presently disclosed subject matter, “a detectably effective amount” of the imaging agent is defined as an amount sufficient to yield an acceptable image using equipment which is available for clinical use. A detectably effective amount of the agent may be administered in more than one injection. The detectably effective amount of the imaging agent can vary according to factors such as the degree of susceptibility of the individual, the age, sex, and weight of the individual, idiosyncratic responses of the individual, the dosimetry, and instrument and film-related factors. Optimization of such factors is well within the level of skill in the art.

The administration of the agent of the invention can be by any means described herein and as generally acceptable to the patient and one of ordinary skill in the art. In particular, the administration of the imaging agent is intravenous.

The predetermined target amount is dependent on a variety of factors including, but not limited to the type of tumor or condition to be observed and the location of any cells desired to be imaged. As such, the predetermined amount is set by one of ordinary skill in the art prior to administration of the agent. Such factors include, but are not limited to, the determined dosage amount, the height, weight, age, body mass index, and gender of the patient or any combination thereof.

In particular embodiments, when the predetermined dosage amount is between about 2.5 mg and about 5.0 mg or greater, the predetermined target amount is 50% of the pre-determined dosage amount. In other embodiments, when the predetermined dosage amount is between about 0.5 mg and about 2.5 mg, the predetermined target amount is 60% of the pre-determined dosage amount. In still other embodiments, when the predetermined dosage amount is about 0.5 mg or less, the predetermined target amount is 80% of the pre-determined dosage amount.

The imaging agent of the invention can be administered by any suitable technique, including both enteral and parenteral methods. In some embodiments, the imaging agents can be formulated into pharmaceutically acceptable formulations and administered intravenously to an organism for imaging. The dosed organism can be imaged using, for example, a FLARE™ Image-Guided Surgery System, which is a continuous-wave (CW) intraoperative imaging system that is capable of simultaneous, real-time acquisition and display of color video (i.e., surgical anatomy) and two channels of invisible NIR fluorescent (700 nm and 800 nm) light. The imaging system can irradiate the dosed organism with radiation absorbed by the imaging agent, and detect optical signals, such as NIR fluorescence, emanating from the targeted portions of the organism containing the imaging agent. The detected signals can be recorded and analyzed by obtaining digital images or video of the subject organism, thereby facilitating diagnostic procedures and image-guided medical techniques.

Any route of administration may be suitable for administering the disclosed agents to a subject. In one embodiment, the disclosed imaging agents may be administered to the subject via intravenous injection. In another embodiment, the disclosed imaging agents may be administered to the subject via any other suitable systemic deliveries, such as parenteral, intranasal, sublingual, rectal, or transdermal administrations.

### Applications, Properties, and Compositions

The conjugates described herein can be used for, e.g., planar optical, optical tomographic, endoscopic, photoacoustic, and sonofluorescent applications for the detection, imaging, and treatment of tumors and other abnormalities. The conjugates can also be used for localized therapy. This can be accomplished, e.g., by directing the conjugates to a desired target site, or allowing the conjugates to accumulate selectively in the target site; shining light of an appropriate wavelength to activate the agent. Thus, the new conjugates can be used to detect, image, and treat a section of tissue, e.g., a tumor.

In addition, the conjugates can be used to detect the presence of tumors and other abnormalities by monitoring the blood clearance profile of the conjugates, for laser assisted guided surgery for the detection of small micrometastases of, e.g., somatostatin subtype 2 (SST-2) positive tumors, and for diagnosis of atherosclerotic plaques and blood clots.

The conjugates can be formulated into diagnostic and therapeutic compositions for enteral or parenteral administration. Generally, these compositions contain an effective amount of the conjugate, along with conventional pharmaceutical carriers and excipients appropriate for the type of administration contemplated. For example, parenteral formulations include the conjugate in a sterile aqueous solution or suspension. Parenteral compositions can be injected directly into a subject at a desired site, or mixed with a large volume parenteral composition for systemic administration. Such solutions can also contain pharmaceutically acceptable buffers and, optionally, electrolytes, such as sodium chloride.

Formulations for enteral administration, in general, can contain liquids, which include an effective amount of the desired dye or dye conjugate in aqueous solution or suspension. Such enteral compositions can optionally include buffers, surfactants, and thixotropic agents. Compositions for oral administration can also contain flavoring agents, and other ingredients for enhancing their organoleptic qualities.

Generally, the diagnostic compositions are administered in doses effective to achieve the desired signal strength to enable detection. Such doses can vary, depending upon the organs or tissues to be imaged, and the imaging equipment being used. For example, Zeheer et al., Nature Biotechnology, 19, 1148-1154 (2001) uses 0.1 mol/kg as a dose for IRDye78 conjugates in vivo. The diagnostic compositions can be administered to a patient systemically or locally to the organ or tissue to be imaged, and then the patient is subjected to the imaging procedure.

Generally, the conjugates or dye compounds absorb and emit light in the visible and infrared region of the electromagnetic spectrum, e.g., they can emit green, yellow, orange, red light, or near infrared light (“NIR”).

In some embodiments, the ZW-800-1, ZW-830-1, ZW-700-1-Forte dye emits and/or absorbs radiation having a wavelength from about 300 nm to about 1000 nm, e.g., from about 400 nm to about 900 nm, or from about 450 mu to about 850 nm. In particular embodiments, the ZW700-1 Forte dye emits and/or absorbs radiation having a wavelength of about 700 nm.

In some embodiments the conjugates and dye compounds have a maximum excitation and/or a maximum emission, measured in 10 mM HEPES solution, pH 7.4, of from about 525 nm to about 875 nm, e.g., from about 550 nm to about 825 nm, or from about 550 nm to about 800 nm.

The invention will be described in greater detail by way of specific examples. The following examples are offered for illustrative purposes and are not intended to limit the invention or claims in any manner. A variety of noncritical parameters in these examples can be changed or modified to yield essentially the same results.

### EXAMPLES

### Example 1: Imaging of Organisms

The FLARE™ Image-Guided Surgery System is a continuous-wave (CW) intraoperative imaging system that is capable of simultaneous, real-time acquisition and display of color video (i.e., surgical anatomy) and two channels of invisible NIR fluorescent (700 nm and 800 nm) light. Details of the theory, engineering, and operation of the imaging system has been described in detail previously. See, Tanaka, E., H. S. Choi, H. Fujii, M. G. Bawendi, and J. V. Frangioni, Image-guided oncologic surgery using invisible light completed: pre-clinical development for sentinel lymph node mapping. Ann Surg Oncol, 2006. 13: 1671-81; De Grand, A. M. and J. V. Frangioni, An operational near-infraredfluorescence imaging system prototype for large animal surgery. Technol Cancer Res Treat, 2003. 2: 553-562; and Nakayama, A., F. del Monte, R. J. Hajjar, and J. V. Frangioni, Functional near-infrared fluorescence imaging for cardiac surgery and targeted gene therapy. Molecular Imaging, 2002. 1: 365-377, each of which is incorporated herein by reference.

Specifications for the FLARE™ Image-Guided Surgery System is provided in Table 1 below.

TABLE 1
FLARE ™ NIR Fluorescence Imaging System Specifications

| Cate [symbol] | Specification | Description |
|---|---|---|
| Physical | Size | Mobile Cart: 32″ W × 32″ D × 41.4″ H; Mast |
| | | Height: 82″ |
| | Weight | 675 lbs, including all electronics |
| | Arm | 6-degree-of-freedom; Reach: 43″-70″ from |
| | | floor, 50.7″ from cart |
| Electrical | Voltage and | 120 V AC, 60 Hz; single NEMA 5-15 120 V/15 |
| | Plug | A AC plug |
| | Current | 15 A max |
| | Grounding | Isolation transformer for all components; |
| | | redundant chassis grounding |
| | Leakage | <300 μA (per AAMI/IEC #60601) |
| Sterility | Shield | Disposable acrylic shield with ≥95% |
| | Drape | Disposable, custom-fit plastic drape bonded to [symbol] |
| Light | Housing | Anodized aluminum with secondary 400 W |
| Source | | cooling plate |
| | Elements | Custom 25 mm circular LED arrays w/ [symbol] |
| | Electronics | Custom passive and active boards with |
| | | embedded controller |
| | Fluence Rates | 40,000 1x white light (400-650 nm), 4 mW/cm² |
| | | of 700 nm (656-678 nm) excitation light, 14 mW/cm² of [symbol] |
| Optics | Working | 18″ from surface of patient |
| | Field-of-View | 2.2 W × 1.7 H cm to 15 W × 11.3 cm (adjustable [symbol] |
| | Emission/ | Color Video (400-650 nm), 700 nm fluorescence |
| | Reflectance Channels | (689-725 nm), 800 nm fluorescence (800-848 nm), all [symbol] |
| | Pixel | 640 × 480 for each camera |
| | System | 125 × 125 μm (x, y) to 625 × 625 μm (x, y) |
| | Display | Up to 15 Hz simultaneous acquisition on all 3 [symbol] |
| | NIR Exposure | Adjustable from 100 μsec to 8 sec |
| Hands | Optics | Automatic zoom/focus |
| Free [symbol] | Control | 6-pedal footswitch |
| Monitors | Number | 2 cart-mounted 20″ for operator; 1 satellite 20″ |
| | | on stand for surgeon |

[symbol]

indicates data missing or illegible when filed

### Example 2: In Vivo Characterization of Dyes and Conjugates

For in vivo characterization, 40 pmol/g (average 10 nmol) of a targeted dye of the invention can be injected IV into 25 g athymic nude mice harboring xenograft human tumors. The FLARE™ imaging system can be set to a 665 nm excitation fluence rate of 1 mW/cm2. Simultaneous color video and NIR fluorescence (700 nm) images can be acquired pre-injection, every 1 sec for the first 20 sec then every 1 min for 2 h. Camera acquisition can be held constant (typically 100 msec) and chosen to ensure that all intensity measurements are within the linear range of the 12-bit Orca-AG (Hamamatsu) NIR camera. Blood can be sampled at 0, 1, 2, 5, 10, 15, 30, 60, and 120 min via tail vein. Intensity-time curves for all major organs and tissues can be quantified. The peak fluorescence intensity and time can be determined for each tumor/tissue/organ, along with the intensity in each at 1 h post-injection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, many equivalents to the specific embodiments of the invention described herein.

Such equivalents are intended to be encompassed by the following claims.

1. An imaging agent dye comprising an imaging agent, optionally conjugated to a targeting vector, the imaging agent dye having one or more N-oxide based zwitterionic groups.

2. The imaging agent dye according to claim 1 wherein the imaging agent is an agent of formula (I): 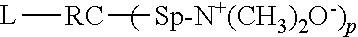
wherein for Formula (I):
L represents a linking group capable of conjugating to a targeting vector;
RC represents a resonant core;
Each Sp independently represents a spacer group; and
p represents an integer from 1-4.

3. The imaging agent dye according to claim 1 wherein the imaging agent is an agent of formula (II): 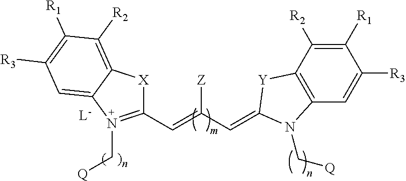
wherein for Formula (II):
each R₁ is independently —C₁-C₄ alkyl-N⁺(CH₃)₂O⁻;
each R₂ is independently H, —OR′, halogen, sulfonate, substituted or unsubstituted amino, C(O)NH—C₁-C₆ alkyl, C₁-C₆ alkyl, C₁-C₆ alkoxy or phenyl;
each R₃ is independently H, OR′, halogen, sulfonate, substituted or unsubstituted amino, C(O)NH—C₁-C₆ alkyl, C₁-C₆ alkyl, C₁-C₆ alkoxy or phenyl;
or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₃)₂O⁻;
X and Y are each independently O, S, Se, C(R″)₂, NR′″;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
each R′ is independently H, alkyl or aryl;
each R″ is independently H or alkyl;
each R′″ is independently H, akyl, akyl-SO₃H, or akyl-COOH;
m is an integer from 0-3; and
each n is independently an integer from 1-4; and
L is an anion;
or a salt, solvate, hydrate, polymorph, prodrug, or stereoisomer thereof;
and wherein the Z group is capable of conjugating a targeting vector.

4. The imaging agent dye according to claim 1 wherein the imaging agent is an agent of formula (III): 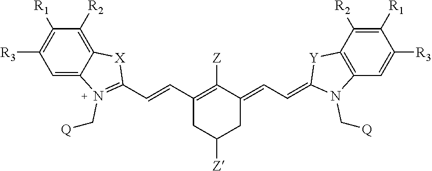
wherein for Formula (III):
each R₁ is independently —C₁-C₄ alkyl-N⁺(CH₃)₂O⁻;
each R₂ is independently H, —OR′, halogen, sulfonato, substituted or unsubstituted amino, C(O)NH—C₁-C₆ alkyl, C₁-C₆ alkyl, C₁-C₆ alkoxy or phenyl;
each R₃ is independently H, —OR′, halogen, sulfonato, substituted or unsubstituted amino, C(O)NH—C₁-C₆ alkyl, C₁-C₆ alkyl, C₁-C₆ alkoxy or phenyl;
or each set of R₁ and R₂ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
or each set of R₁ and R₃ which is bound to the same ring can be taken together with the carbon atoms to which they are attached to form a 5-6 membered aryl or heteroaryl ring substituted with at least one —C₁-C₄ alkyl-N⁺(CH₃)₂O⁻ group, and, optionally, further substituted with halogen, alkyl, alkoxy, hydroxyl, —SO₂OH, or —CO₂H;
each Q is N⁺(CH₃)₂O⁻;
X and Y are each independently O, S, Se, C(R″)₂, NR;
Z is H, halogen, CN, R₆, OR₆, SR₆, NHR₆ or CH₂R₆, in which R₆ is optionally substituted C₁-C₆ alkyl, optionally substituted aryl, or optionally substituted heteroaryl, alkyl-N₃, aryl-N₃, aryl-halogen;
Z′ is H, or COOR;
R is independently H, OR″″, each R′ is independently H, alkyl or aryl;
each R″ is independently H or alkyl;
each R′″ is independently H, akyl, akyl-SO₃H, or akyl-COOH;
each R″″ is independently H, akyl, or aryl, NH₂, NHR, alkyl-NH₂, or alkyl-COOH;
each n is independently an integer from 1-4; and
L is an anion;
or a salt, solvate, hydrate, polymorph, prodrug, or stereoisomer thereof;
and wherein the Z group is capable of conjugating a targeting vector.

5. The imaging agent dye according to claim 1, wherein the imaging agent is:

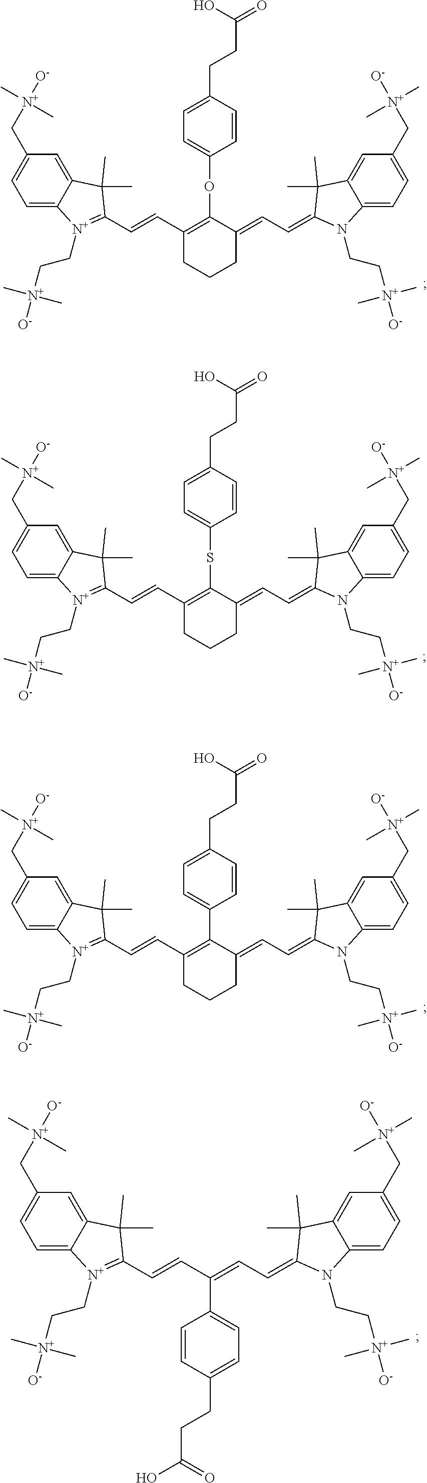

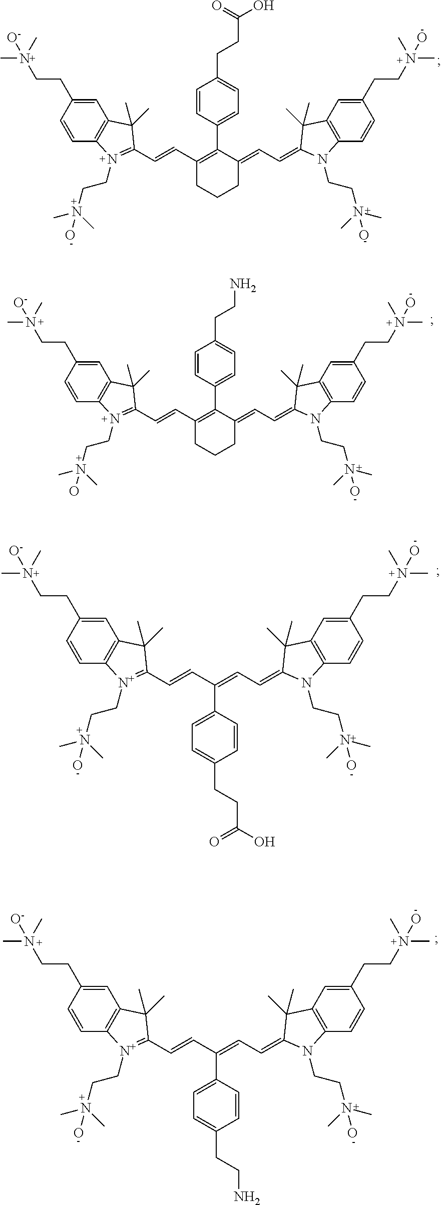

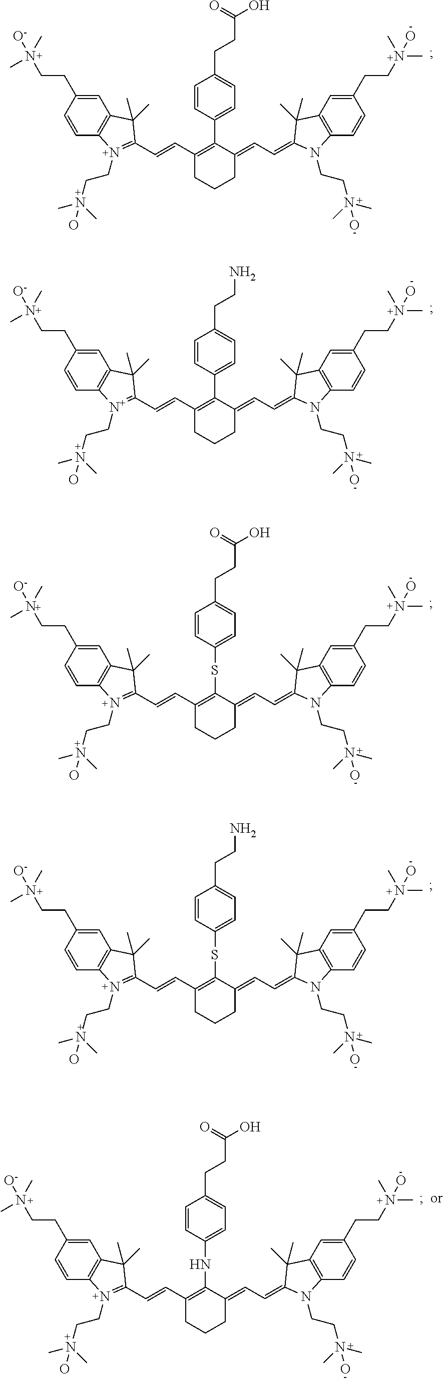

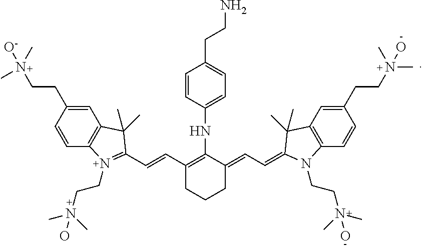

6. The imaging agent dye according to claim 1 wherein the targeting vector is cRGD, dPSMA-617, KUE, a FAP binding vector, octreotide, bombesin, or a GPI-derivative.

7. The imaging agent dye according to claim 1 wherein the imaging agent is conjugated to the targeting vector via a direct bond, or via a linking group.

8. A method of imaging tissue, cells, or lumen in a subject, the method comprising:
(a) contacting the tissue, cells or lumen with an imaging agent comprising a dye comprising an imaging agent according to claim 1 to the subject,
(b) irradiating the tissue, cells, or lumen at a wavelength absorbed by the dye;
(c) and detecting a signal from the imaging agent, thereby imaging the tissue, cells, or lumen.

9. A method of imaging tissue, cells, or lumen in a subject according to claim 8, wherein the cells are tumor cells.

10. The method of claim 8, wherein the subject is human.

11. The method of claim 8, wherein the imaging agent has peak absorbance at about 600 nm to 850 nm.

12. The method of claim 8, wherein the tissue or cells is imaged in vivo.

13. The method of claim 8, wherein the imaging agent further comprises a PEG-moiety.

14. The method of claim 8, wherein the imaging agent further comprises a radioisotope for either single-photon emission computed tomography (SPECT) or positron emission tomography (PET).

15. The method of claim 8, wherein the imaging agent comprises a reactive linking group, such as NHS ester, sulfo-NHS ester, or a TFP ester.

16. A method of treating cancer in a subject, the method comprising:
(a) administering an imaging effective amount of an imaging agent according to claim 1 to a subject,
(b) irradiating the cells, tissues or organs of a subject suspected of being cancerous at a wavelength absorbed by the imaging agent;
(c) diagnosing the cancer in the cells tissues, or organs of the subject by detecting a signal from the imaging agent; and
(d) administering a chemotherapeutic treatment, a radiotherapeutic treatment, or a surgical treatment to the subject to treat the cancer.


SEQUENCE LISTING (6 sequences)

SEQ ID NO: 1 - protein, 9 aa, Homo sapiens
CGQKRTRGC

SEQ ID NO: 2 - protein, 12 aa, Homo sapiens
HTMYYHHYQHHL

SEQ ID NO: 3 - protein, 9 aa, Homo sapiens
CRKRLDRNC

SEQ ID NO: 4 - protein, 6 aa, Homo sapiens
CSPGAK

SEQ ID NO: 5 - protein, 8 aa, Homo sapiens
FCFWKTCT

SEQ ID NO: 6 - protein, 14 aa, Homo sapiens
XQRLGNQWAVGHLM